(19) United States
(12) Patent Application Publication
(10) Pub. No.: US 2026/0321356 A1
(43) Pub. Date: Oct. 15, 2026

(54) MULTI-DIMENSIONAL DATA VISUALIZATION FOR MULTIPLEXED SAMPLE QUANTIFICATION

(71) Applicant: Life Technologies Corporation, Carlsbad, CA (US)

(72) Inventors: Nivedita Sumi Majumdar, Martinez, CA (US); Sitong Liu, Santa Clara, CA (US)

(21) Appl. No.: 19/475,355

(22) PCT Filed: Apr. 19, 2024
(86) PCT No.: PCT/US2024/025476

§ 371 Date: Oct. 14, 2025

Related U.S. Application Data
(60) Provisional application No. 63/497,403, filed on Apr. 20, 2023.

(51) Int. Cl. G16B 45/00; G16B 25/20

(52) U.S. Cl. CPC G16B 45/00; G16B 25/20

(57) ABSTRACT

A computer-implemented method for visualizing dye interaction in a multiplexed biological sample is provided. The method includes receiving fluorescent emission data from each reaction site of a plurality of reaction sites. The reaction sites include at least a first, second, and third dye. The method further includes determining intensity values for at least a first, second, and third dye channel from the fluorescent emission data from each reaction site. The method further includes displaying, on a user interface, a first set of indications of a plurality of intensity values of the fluorescent emission data for the first, second, and third dye detected in the first dye channel, detected in the second dye channel, and detected in the third dye channel. The method further includes adjusting a label of the fluorescent emission data from a reaction site by comparing the first set of indications.

### BACKGROUND

Systems for biological and biochemical reactions have been used to monitor, measure, and/or analyze such reactions in real time. Such systems are commonly used in sequencing, genotyping, polymerase chain reaction (PCR), and other biochemical reactions to monitor the progress and provide quantitative data.

Currently, there is an increasing demand to provide greater numbers of reactions per test or experiment have resulted in instruments that are able to conduct ever higher numbers of reactions simultaneously. The increase in the number sample sites in a test or experiment has led to microtiter plates and other sample formats that provide ever smaller sample volumes. In addition, techniques such as digital PCR (dPCR) have increased the demand for smaller sample volumes that contain either zero or one target nucleotide sequence in all or the majority of a large number of test samples.

Digital PCR may be used to detect and quantify the concentration of rare alleles, to provide absolute quantitation of nucleic acid samples, and to measure low fold-changes in nucleic acid concentration. Generally, increasing the number of replicates increases the accuracy and reproducibility of dPCR results.

In dPCR, a solution containing a relatively small number of a target polynucleotide or nucleotide sequence may be subdivided into a large number of small test samples, such that each sample generally contains either one molecule of the target nucleotide sequence or none of the target nucleotide sequence. When the samples are subsequently thermally cycled in a PCR protocol, procedure, or experiment, the samples containing the target nucleotide sequence are amplified and produce a positive detection signal, while the samples containing no target nucleotide sequence are not amplified and produce no detection signal. A fluorescent intensity threshold is used by a processor to determine which samples are considered positive and negative detection.

However, in samples using multiple dyes, chemical interaction gives rise to some non-specific amplification creating a challenge of how to define the threshold fluorescence.

Furthermore, the immense number of data points the data collected from a dPCR experiment is challenging to organize and visualize in a manner that is useful to a user.

Typically, 2D scatter plot data visualizations are generated to view the fluorescent intensity detection and to adjust the threshold and make determinations about positive and negative fluorescent detection calls. However, scatter plots only allow a visualization of data of one or two dyes. It does not allow visualization of more than two dyes and interactions between multiple dyes. Spectral interaction between dyes may cause the data clusters displayed in the scatter plot to shift or subclusters to form when more than one target has positive amplification. Thus, typical 2D scatter plots do not help a user determine better positive and negative calls to improve dPCR results.

As such, a data visualization that gives the ability to investigate spectral and chemical interactions and allows flexible positive and negative detection calling in a multi-dimensional space is needed.

### SUMMARY

In one exemplary embodiment, a computer-implemented method for visualizing dye interaction in a multiplexed biological sample is provided. The method includes receiving fluorescent emission data from each reaction site of a plurality of reaction sites, where the plurality of reaction sites include at least a first, second, and third dye. The method further includes determining intensity values for at least a first, second, and third dye channel from the fluorescent emission data from each reaction site of the plurality of reaction sites. The method further includes displaying, on a user interface, a first set of indications of a plurality of intensity values of the fluorescent emission data for the first, second, and third dye detected in the first dye channel, displaying, on the user interface, a second set of indications of a plurality of intensity values of the fluorescent emission data for the first, second, and third dye detected in the second dye channel, and displaying, on the user interface, a third set of indications of a plurality of intensity values of the fluorescent emission data for the first, second, and third dye detected in the third dye channel. The method further includes adjusting a label of the fluorescent emission data from a reaction site by comparing the first set of indications.

In another exemplary embodiment, a system for visualizing dye interaction in a multiplexed biological sample is provided. The system includes a plurality of reaction sites, wherein each reaction site includes a biological sample, a detector configured to receive fluorescent emission data from the plurality of reaction sites, a processor configured to determine intensity values for at least a first, second, and third dye channel from the fluorescent emission data from each reaction site of the plurality of reaction sites, and a user interface. The user interface is configured to display first set of indications of a plurality of intensity values of the fluorescent emission data for the first, second, and third dye detected in the first dye channel, display a second set of indications of a plurality of intensity values of the fluorescent emission data for the first, second, and third dye detected in the second dye channel, and display a third set of indications of a plurality of intensity values of the fluorescent emission data for the first, second, and third dye detected in the third dye channel.

In yet another exemplary embodiment, a computer-readable medium encoded with computer-readable instructions is provided. The computer-readable instructions, which when executed by a processor of a computer, causes the computer to carry out a method for visualizing dye interaction in a multiplexed biological sample. The method includes receiving fluorescent emission data from each reaction site of a plurality of reaction sites, where the plurality of reaction sites include at least a first, second, and third dye. The method further includes determining intensity values for at least a first, second, and third dye channel from the fluorescent emission data from each reaction site of the plurality of reaction sites. The method further includes displaying, on a user interface, a first set of indications of a plurality of intensity values of the fluorescent emission data for the first, second, and third dye detected in the first dye channel, displaying, on the user interface, a second set of indications of a plurality of intensity values of the fluorescent emission data for the first, second, and third dye detected in the second dye channel, and displaying, on the user interface, a third set of indications of a plurality of intensity values of the fluorescent emission data for the first, second, and third dye detected in the third dye channel. The method further includes adjusting a label of the fluorescent emission data from a reaction site by comparing the first set of indications.

### DESCRIPTION OF THE FIGURES

FIG. 1 illustrates a flowchart showing a method of generating a multi-dimensional data visualization according to various embodiments described herein.

FIG. 2 illustrates an exemplary computing system that various embodiments described herein may be implemented.

FIG. 3 is a block diagram that illustrates a polymerase chain reaction (PCR) instrument, upon which embodiments of the present teachings may be implemented.

FIG. 4 illustrates an exemplary optics system that can be used to image the chip according to embodiments of the present teachings.

FIG. 5 illustrates a chip including reaction sites where data is gathered from and visualized according to various embodiments described herein.

FIGS. 6A and 6B illustrate a data visualization of fluorescent intensity of a plurality of reaction sites according to various embodiments described herein.

FIG. 7 illustrates a 2D scatter plot data visualization according to various embodiments described herein.

FIGS. 8A, 8B, 8C, and 8D illustrate data visualizations of fluorescent intensity of a plurality of reaction sites according to various embodiments described herein.

FIG. 9 illustrates a 2D scatter plot data visualization according to various embodiments described herein

FIG. 10 illustrates a 2D scatter plot data visualization according to various embodiments described herein

FIG. 11 illustrates a multi-dimensional data visualization according to various embodiments described herein.

FIG. 12 illustrates a multi-dimensional data visualization according to various embodiments described herein.

FIG. 13 illustrates a user interface according to various embodiments described herein.

FIG. 14 illustrates a user interface according to various embodiments described herein.

FIG. 15A illustrates a 2D scatter plot data visualization according to various embodiments described herein.

FIG. 15B illustrates a corresponding multi-dimensional data visualization to FIG. 15A according to various embodiments described herein.

FIG. 16 illustrates a selection menu of a user interface according to various embodiments described herein.

FIGS. 17A and 17B illustrate tools of a user interface according to various embodiments described herein.

FIG. 18 illustrates a user interface for a 2D scatter plot data visualization according to various embodiments described herein.

FIG. 19 illustrates a 2D scatter plot data visualization according to various embodiments described herein.

FIG. 20 illustrates a multi-dimensional data visualization according to various embodiments described herein.

FIG. 21 illustrates a 2D scatter plot data visualization according to various embodiments described herein.

FIG. 22 illustrates a multi-dimensional data visualization according to various embodiments described herein.

FIG. 23 illustrates a 2D scatter plot data visualization according to various embodiments described herein.

FIG. 24 illustrates a multi-dimensional data visualization according to various embodiments described herein.

FIG. 25 illustrates a 2D scatter plot data visualization according to various embodiments described herein.

FIG. 26 illustrates a multi-dimensional data visualization according to various embodiments described herein.

### DETAILED DESCRIPTION

To provide a more thorough understanding of the present invention, the following description sets forth numerous specific details, such as specific configurations, parameters, examples, and the like. It should be recognized, however, that such description is not intended as a limitation on the scope of the present invention but is intended to provide a better description of the exemplary embodiments.

As described above, biological analysis often involves many replicates to improve accuracy. It is challenging to understand the immense amount of data that is received and how to use the data to determine accurate results. Thus, data visualizations that help a user understand the data and make adjustments to generate better results are needed.

For example, oncology assays often have chemical crosstalk, or non-specific binding, between replicates. This results in difficulty in determining whether a reaction site would be considered positive for dye fluorescence. A single intensity threshold to determine a positive or negative call may not lead to a more accurate result.

Furthermore, if multiple target dyes are used in an assay, the interaction between the dyes cannot be determined. Previously used 2D data visualizations would not show the whole picture of interactions between three, four, five, etc. dyes. A combination of several 2D view would be needed to understand the full picture of what is happening.

As such, according to embodiments described herein, generation of a multi-dimensional data visualization for viewing multiplex data is needed to generate better and more accurate results from biological assays.

Various embodiments described herein are particularly suited for digital PCR (dPCR). In digital PCR, a solution containing a relatively small number of a target polynucleotide or nucleotide sequence may be subdivided into a large number of small test samples, such that each sample generally contains either one molecule of the target nucleotide sequence or none of the target nucleotide sequence. When the samples are subsequently thermally cycled in a PCR protocol, procedure, or experiment, the sample containing the target nucleotide sequence are amplified and produce a positive detection signal, while the samples containing no target nucleotide sequence are not amplified and produce no detection signal. Using Poisson statistics, the number of target nucleotide sequences in the original solution may be correlated to the number of samples producing a positive detection signal.

In order to conduct a typical dPCR protocol, procedure, or experiment, it is advantageous to be able to divide an initial sample solution into tens of thousands or hundreds of thousands of test samples each having a volume of several nanoliters, at or about one nanoliter, or less than one nanoliter, in a way that is simple and cost effective. Because the number of target nucleotide sequences may be very small, it may also be important in such circumstances that the entire content of the initial solution be accounted for and contained in the plurality of reaction sites.

In various embodiments, the devices, instruments, systems, and methods described herein may be used to detect one or more types of biological components of interest. These biological components of interest may include, but are not limited to, DNA sequences, RNA sequences, genes, oligonucleotides, or cells (e.g., circulating tumor cells). In various embodiments, such biological components may be used in conjunction with various PCR, qPCR, and/or dPCR methods and systems in applications such as fetal diagnostics, multiplex dPCR, viral detection and quantification standards, genotyping, sequencing validation, mutation detection, detection of genetically modified organisms, rare allele detection, and copy number variation.

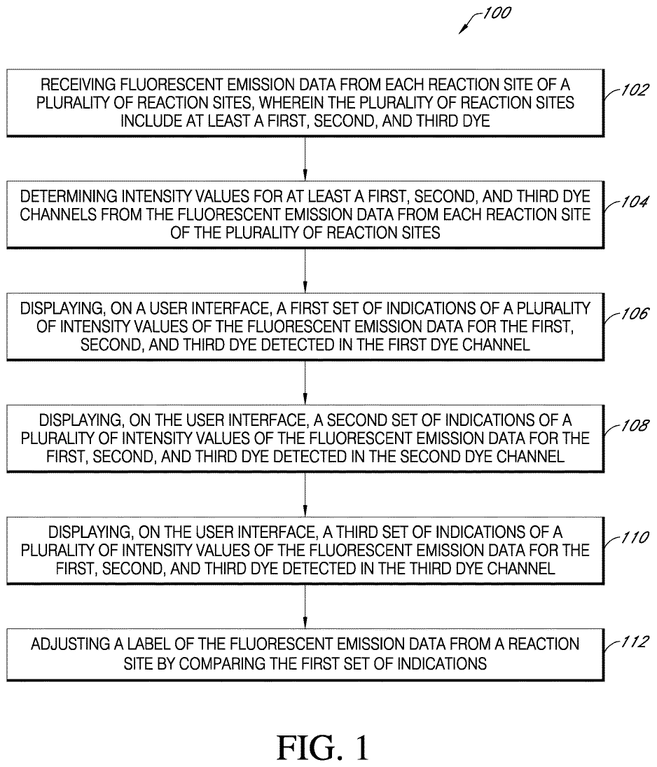

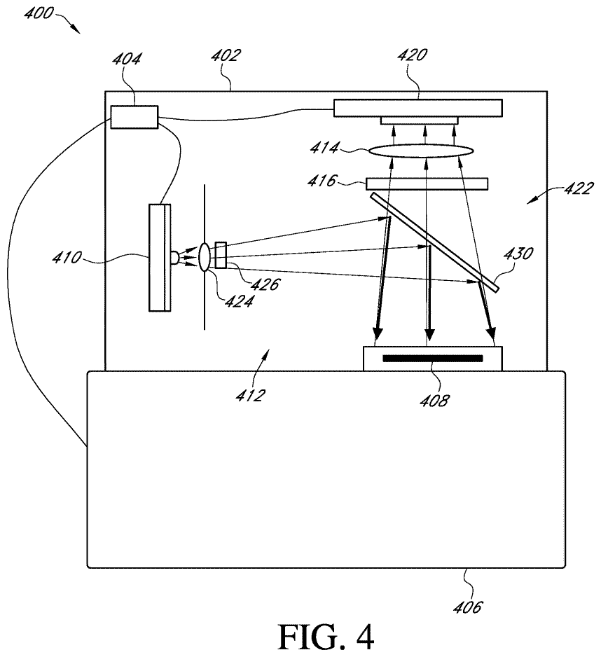

FIG. 1 illustrates a flowchart of method 100 for generating a multi-dimensional data visualization for visualizing dye interaction in a multiplexed biological sample according to various embodiments described herein. Step 102 is receiving fluorescent emission data from each reaction site of a plurality of reaction sites, where the plurality of reaction sites include at least a first, second, and third dye. An optical system, such as one described with reference to FIG. 4, is used to detect fluorescent emissions from reaction sites. A multiplex assay may use more than two dyes. According to various embodiments, three, four, or five, or more dyes may be used. In step 104, intensity values for at least a first, second, and third dye channels from the fluorescent emission data from each reaction site of the plurality of reaction sites are determined.

Method 100 further includes step 106 in which, a first set of indications of a plurality of intensity values of the fluorescent emission data for the first, second, and third dye detected in the first dye channel is displayed on a user interface. Indications, according to embodiments of the present teachings, may include, colors, shapes, colored shapes, labels, or patterned lines, for example. An indication, according to various embodiments, is a visual indication that allows a user to distinguish between data sets. For example, a single color may be used to show the data initially determined to be positive for fluorescence. Indications of a plurality of intensity values include lines joining the intensity values to the next intensity value in the next dye channel.

According to various embodiments, joining the intensity values by lines help show the change of the same data emission data in the different dye channel. Indications of a plurality of intensity values may be viewed as data bands across the dye channels. The changing of the data band from channel to channels gives valuable information to a user that would not be understood by a user using only a 2D scatter plot data visualization.

Intensity data of the fluorescent emission data include data from the three dyes used in the assay and detected in a first dye channel is displayed on a user interface. In this way, the differences in fluorescent intensity may be compared between different categories of data.

Fluorescence due to crosstalk or spectral interaction may be more easily visualized and adjustments may be made that affect the results of the assay. As an example, the first dye channel may be FAM.

Similarly, in step 108, a second set of indications of a plurality of intensity values of the fluorescent emission data for the first, second, and third dye detected in the second dye channel is displayed on the user interface. Following the example, the second dye channel may be VIC.

Step 110 is displaying, on the user interface, a third set of indications of a plurality of intensity values of the fluorescent emission data for the first, second, and third dye detected in the third dye channel. Following the example, the third dye channel may be ABY.

Method 100 further includes step 112 of adjusting a label of the fluorescent emission data from a reaction site by comparing the first set of indications. A label could be FAM positive detection, but upon reviewing the multi-dimensional data visualization, according to various embodiments, described herein, it can be determined that detected fluorescence may have been due to spectral interaction and the label is adjusted to FAM negative detection. Adjusting the label can then change other data visualizations generated for this assay, resulting in more accurate different visualizations to be generated. Further, an ultimate result for the assay, such as concentration, may also be generated and improved from the more accurately called data.

For example, changing a label in the multi-dimensional data visualization may result in a generation of a new or improved 2D scatter plot. The scatter plot may include adjusted negative or positive calls and can now generate a more accurate result. Similarly, a change in a 2D scatter plot may also generate a change in the multi-dimensional data visualization.

Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic. (The term “logic” herein refers to fixed hardware, programmable logic and/or an appropriate combination thereof, as would be recognized by one skilled in the art to carry out the recited functions.) Software and firmware can be stored on computer-readable media. Some other processes can be implemented using analog circuitry, as is well known to one of ordinary skill in the art. Additionally, memory or other storage, as well as communication components, may be employed in embodiments of the invention.

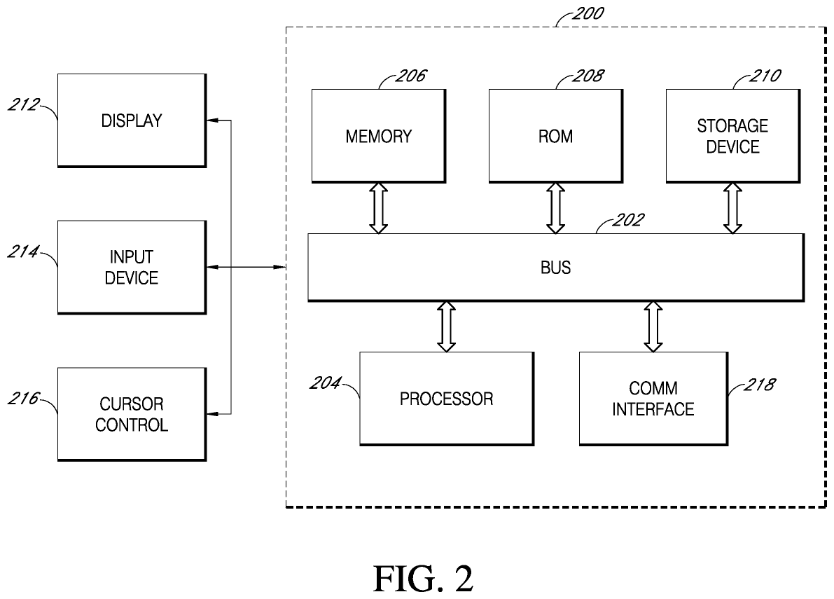

FIG. 2 is a block diagram that illustrates a computer system 200 that may be employed to carry out processing functionality, according to various embodiments, upon which embodiments of a thermal cycler system (FIG. 2) may utilize. Computing system 200 can include one or more processors, such as a processor 204. Processor 204 can be implemented using a general or special purpose processing engine such as, for example, a microprocessor, controller or other control logic. In this example, processor 204 is connected to a bus 202 or other communication medium.

Further, it should be appreciated that a computing system 200 of FIG. 2 may be embodied in any of a number of forms, such as a rack-mounted computer, mainframe, supercomputer, server, client, a desktop computer, a laptop computer, a tablet computer, hand-held computing device (e.g., PDA, cell phone, smart phone, palmtop, etc.), cluster grid, netbook, embedded systems, or any other type of special or general purpose computing device as may be desirable or appropriate for a given application or environment. Additionally, a computing system 200 can include a conventional network system including a client/server environment and one or more database servers, or integration with LIS/LIMS infrastructure. A number of conventional network systems, including a local area network (LAN) or a wide area network (WAN), and including wireless and/or wired components, are known in the art. Additionally, client/server environments, database servers, and networks are well documented in the art.

Computing system 200 may include bus 202 or other communication mechanism for communicating information, and processor 204 coupled with bus 202 for processing information.

Computing system 200 also includes a memory 206, which can be a random-access memory (RAM) or other dynamic memory, coupled to bus 202 for storing instructions to be executed by processor 204. Memory 206 also may be used for storing temporary variables or other intermediate information during execution of instructions to be executed by processor 204. Computing system 200 further includes a read only memory (ROM) 208 or other static storage device coupled to bus 202 for storing static information and instructions for processor 204.

Computing system 200 may also include a storage device 210, such as a magnetic disk, optical disk, or solid-state drive (SSD) is provided and coupled to bus 202 for storing information and instructions. Storage device 210 may include a media drive and a removable storage interface. A media drive may include a drive or other mechanism to support fixed or removable storage media, such as a hard disk drive, a floppy disk drive, a magnetic tape drive, an optical disk drive, a CD or DVD drive (R or RW), flash drive, or other removable or fixed media drive. As these examples illustrate, the storage media may include a computer-readable storage medium having stored therein particular computer software, instructions, or data.

In alternative embodiments, storage device 210 may include other similar instrumentalities for allowing computer programs or other instructions or data to be loaded into computing system 200. Such instrumentalities may include, for example, a removable storage unit and an interface, such as a program cartridge and cartridge interface, a removable memory (for example, a flash memory or other removable memory module) and memory slot, and other removable storage units and interfaces that allow software and data to be transferred from the storage device 210 to computing system 200.

Computing system 200 can also include a communications interface 218. Communications interface 218 can be used to allow software and data to be transferred between computing system 200 and external devices. Examples of communications interface 218 can include a modem, a network interface (such as an Ethernet or other NIC card), a communications port (such as for example, a USB port, a RS-232C serial port), a PCMCIA slot and card, Bluetooth, etc. Software and data transferred via communications interface 218 are in the form of signals which can be electronic, electromagnetic, optical or other signals capable of being received by communications interface 218. These signals may be transmitted and received by communications interface 218 via a channel such as a wireless medium, wire or cable, fiber optics, or another communications medium. Some examples of a channel include a phone line, a cellular phone link, an RF link, a network interface, a local or wide area network, and other communications channels.

Computing system 200 may be coupled via bus 202 to a display 212, such as a cathode ray tube (CRT) or liquid crystal display (LCD), for displaying information to a computer user.

An input device 214, including alphanumeric and other keys, is coupled to bus 202 for communicating information and command selections to processor 204, for example. An input device may also be a display, such as an LCD display, configured with touchscreen input capabilities. Another type of user input device is cursor control 216, such as a mouse, a trackball or cursor direction keys for communicating direction information and command selections to processor 204 and for controlling cursor movement on display 212. This input device typically has two degrees of freedom in two axes, a first axis (e.g., x) and a second axis (e.g., y), that allows the device to specify positions in a plane. A computing system 200 provides data processing and provides a level of confidence for such data. Consistent with certain implementations of embodiments of the present teachings, data processing and confidence values are provided by computing system 200 in response to processor 204 executing one or more sequences of one or more instructions contained in memory 206. Such instructions may be read into memory 206 from another computer-readable medium, such as storage device 210.

Execution of the sequences of instructions contained in memory 206 causes processor 204 to perform the process states described herein. Alternatively, hard-wired circuitry may be used in place of or in combination with software instructions to implement embodiments of the present teachings. Thus, implementations of embodiments of the present teachings are not limited to any specific combination of hardware circuitry and software.

The term “computer-readable medium” and “computer program product” as used herein generally refers to any media that is involved in providing one or more sequences or one or more instructions to processor 204 for execution. Such instructions, generally referred to as “computer program code” (which may be grouped in the form of computer programs or other groupings), when executed, enable the computing system 200 to perform features or functions of embodiments of the present invention. These and other forms of computer-readable media may take many forms, including but not limited to, non-volatile media, volatile media, and transmission media. Non-volatile media includes, for example, solid state, optical or magnetic disks, such as storage device 210. Volatile media includes dynamic memory, such as memory 206. Transmission media includes coaxial cables, copper wire, and fiber optics, including the wires that comprise bus 202.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 204 for execution. For example, the instructions may initially be carried on magnetic disk of a remote computer. The remote computer can load the instructions into its dynamic memory and send the instructions over a telephone line using a modem. A modem local to computing system 200 can receive the data on the telephone line and use an infra-red transmitter to convert the data to an infra-red signal. An infra-red detector coupled to bus 202 can receive the data carried in the infra-red signal and place the data on bus 202. Bus 202 carries the data to memory 206, from which processor 204 retrieves and executes the instructions. The instructions received by memory 206 may optionally be stored on storage device 210 either before or after execution by processor 204.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

In various embodiments, the devices, instruments, systems, and methods described herein may be used to detect one or more types of biological components of interest. These biological components of interest may be any suitable biological target including, but are not limited to, DNA sequences (including cell-free DNA), RNA sequences, genes, oligonucleotides, molecules, proteins, biomarkers, cells (e.g., circulating tumor cells), or any other suitable target biomolecule.

In various embodiments, such biological components may be used in conjunction with various PCR, qPCR, and/or dPCR methods and systems in applications such as fetal diagnostics, multiplex dPCR, viral detection and quantification standards, genotyping, sequencing validation, mutation detection, detection of genetically modified organisms, rare allele detection, and copy number variation. Embodiments of the present disclosure are generally directed to devices, instruments, systems, and methods for monitoring or measuring a biological reaction for a large number of small volume samples. As used herein, samples may be referred to as sample volumes, or reactions volumes, for example.

While generally applicable to quantitative polymerase chain reactions (qPCR) where a large number of samples are being processed, it should be recognized that any suitable PCR method may be used in accordance with various embodiments described herein. Suitable PCR methods include, but are not limited to, digital PCR, allele-specific PCR, asymmetric PCR, ligation-mediated PCR, multiplex PCR, nested PCR, qPCR, genome walking, and bridge PCR, for example.

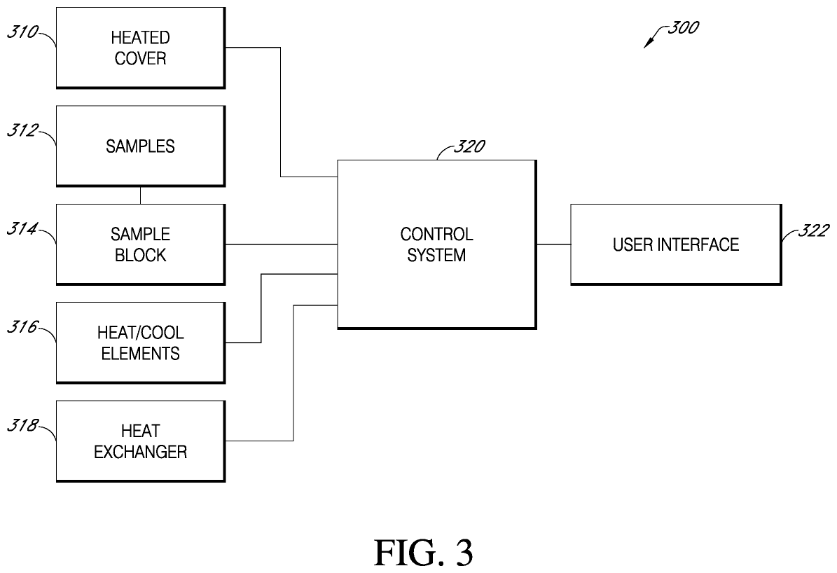

For embodiments of PCR instrument 300 in FIG. 3, control system 320, may be used to control the functions of the detection system, heated cover, and thermal block assembly. Control system 320 may be accessible to an end user through user interface 322 of PCR instrument 300 in FIG. 3. Also a computing system 200, as depicted in FIG. 2, may serve as to provide the control the function of PCR instrument 300 in FIG. 3, as well as the user interface function. Additionally, computing system 200 of FIG. 2 may provide data processing, display and report preparation functions. All such instrument control functions may be dedicated locally to the PCR instrument, or computer system 200 of FIG. 2 may provide remote control of part or all of the control, analysis, and reporting functions, as will be discussed in more detail subsequently. Instrument control functions may be provided on the instrument, accessible through a graphical user interface (GUI). Further, in various embodiments, data analysis controls may be provided on the instrument, accessible through a GUI. In various embodiments, data analysis of the results of the system may be performed at a local computer system, connected to the instrument. In other embodiments, data analysis functions may be accessed over a network by a user. Data from performing biological reactions by the system, according to various embodiments, may be stored on a server system to be accessible by users over a network.

As mentioned above, an instrument that may be utilized according to various embodiments, but is not limited to, is a polymerase chain reaction (PCR) instrument. FIG. 3 is a block diagram that illustrates a PCR instrument 300, upon which embodiments of the present teachings may be implemented. PCR instrument 300 may include a heated cover 310 that is placed over a plurality of samples 312 contained in a sample support device (not shown). In various embodiments, a sample support device may be a chip, or glass or plastic slide with a plurality of reaction sites, which reaction sites have a cover between the reaction sites and heated cover 310. Some examples of a sample support device may include, but are not limited to, a chip according to embodiments of the present teachings, a multi-well plate, such as a standard microtiter 96-well, a 384-well plate, or a microcard, or a substantially planar support, such as a glass or plastic slide. The reaction sites in various embodiments of a sample support device may include depressions, indentations, ridges, and combinations thereof, patterned in regular or irregular arrays formed on the surface of the substrate.

Various embodiments of PCR instruments include a sample block 314, elements for heating and cooling 316, a heat exchanger 318, control system 320, and user interface 322.

Various embodiments of a thermal block assembly according to the present teachings comprise components 314-318 of PCR instrument 300 of FIG. 3.

According to other embodiments of the present teachings, the thermal block assembly includes thermal electric devices such that substantial uniform heat transfer is provided throughout the thermal block assembly.

As mentioned above, detection of the target may include fluorescence detection, detection of positive or negative ions, pH detection, voltage detection, or current detection, for example. As such, a detection system, according to various embodiments described herein may include an optical system, an electrical detection system, an ion detection system, or a pH detection system, for example. According to various embodiments, the detection system may be integrated in the chip.

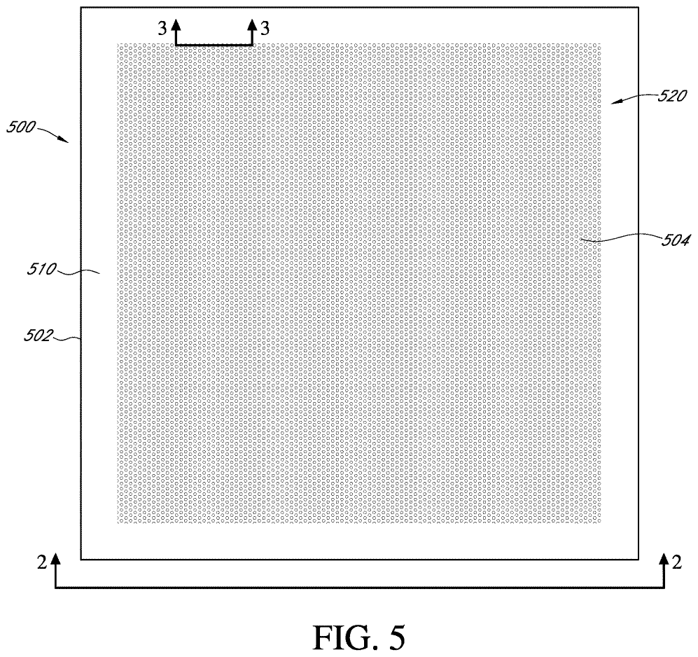

Referring to FIG. 4, as mentioned above, a system 400 may be used optically view, inspect, detect, or measure one or more targets contained in the reaction sites. Reaction sites can including in a chip 408, which may be contained in a carrier. Chip 408 may be chip 500 (FIG. 5) according to various embodiments. System 400 comprises an optical head or system 402.

System 400 may further comprise a controller, computer, or processor 404 configured, for example, to operate various components of optical system 402 or to obtain and/or process data provided by system 400. For example, processor 404 may be used to obtain and/or process optical data provided by one or more photodetectors of optical system 402. In other embodiments, processor 404 may transmit data to one or more computing systems for further processing. Data may be transmitted from processor 404 to the computing systems, via a network, in some embodiments.

In certain embodiments, system 400 further comprises a thermal control system 406 comprising, for example, a thermal cycler configured to perform a PCR procedure or protocol on at least some of the samples contained in chip 408. Systems 402, 406 may combined or coupled together into a single unit, for example, in order to perform a qPCR and/or a dPCR procedure or protocol on at least some of the samples contained in chip 408. In such embodiments, computer 404 may be used to control systems 402, 406 and/or to collect or process data provided or obtained by either or both systems 402, 406. In other embodiments, system 402 and system 406 may be independent units.

In certain embodiments, optical system 402 comprises a light source 410 and an associated excitation optic system 412 configured to illuminate at least some of samples contained in the reaction sites of chip 408. Excitation optical system 412 may include one or more lenses 414 and/or one or more filters 416 for conditioning light directed to the samples. Optical system 402 may further comprise a photodetector 420 and an associated emission optic system 422 configured to receive optical data emitted by at least some of samples contained in the reaction sites of chip 408. For example, when system 400 is configured to perform a qPCR and/or a dPCR procedure, the sample may contain fluorescent dyes that provide a fluorescent signal that varies according to an amount of target nucleotide sequence contained in various of the through-holes of chip 408. Emission optical system 422 may include one or more lenses 424 and/or one or more filters 426 for conditioning light directed to the samples.

According to various embodiments, optical system 402 may have a focal length of 15 mm and a working distance of 60 mm, where the working distance of the distance from the chip to the camera lens. Furthermore, in various embodiments, the overall system F-number is less than or equal to 3.

In the illustrated embodiment of FIG. 4, excitation/emission optical systems 412, 422 both comprise one or more common optical elements. For example, excitation/emission optical systems 412, 422 both comprise a beamsplitter 430 that reflects excitation light and transmits emission light from the samples to photodetector 420. In certain embodiments, excitation/emission optical systems 412, 422 both comprise a field lens (not shown) disposed between beamsplitter 430 and chip 408, which may be used improve optical performance, for example, to provide more even illumination and reading of light to and from the samples contained in chip 408. In certain embodiments, for example where even illumination is less critical (e.g., some dPCR applications), the common field lens may be omitted, as shown in the illustrated embodiment of FIG. 4. Omission of the field lens may help to reduce the size and complexity of optical system 402.

As described below, in accordance with various embodiments described herein, reaction sites may include, but are not limited to, through-holes, wells, indentations, spots, cavities, sample retainment regions, and reaction chambers, for example.

Furthermore, as used herein, thermal cycling may include using a thermal cycler, isothermal amplification, thermal convention, infrared mediated thermal cycling, or helicase dependent amplification, for example. In some embodiments, the chip may be integrated with a built-in heating element. In various embodiments, the chip may be integrated with semiconductors.

According to various embodiments, detection of a target may be, but is not limited to, fluorescence detection, detection of positive or negative ions, pH detection, voltage detection, or current detection, alone or in combination, for example.

With reference to FIG. 5, in certain embodiments of the present teachings a chip 500 comprises a substrate 502 and a plurality of reaction sites. Chip 500 may also be referred to as an article, device, array, slide, or platen, for example.

According to various embodiments of the present disclosure, reaction sites may be, but are not limited to, wells, cavities, indentations, spots, reaction chambers, sample retainment regions, or through-holes, for example, located in substrate 502. Reaction sites may be any structure that allows a sample to be independent of other samples located on the substrate. Substrate 502 comprises a first surface 510 and an opposing second surface 512.

The reactions sites 504 are configured to provide sufficient surface tension by capillary action to hold respective liquid samples containing a biological sample to be processed or examined.

Substrate 502 may be a flat plate or comprise any form suitable for a particular application or design. Substrate may comprise, in total or in part, any of the various materials known in the fabrication arts including, but not limited to, a metal, glass, ceramic, silicon material, or the like. Additionally, or alternatively, substrate 502 may comprise a polymer material such as an acrylic, styrene, polyethylene, polycarbonate, and polypropylene material. Substrate 502 and reaction sites 504 may be formed by one or more of machining, injection molding, hot embossing, laser drilling, photolithography, or the like.

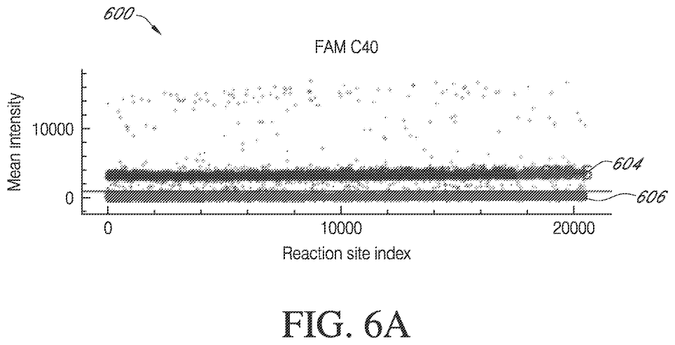

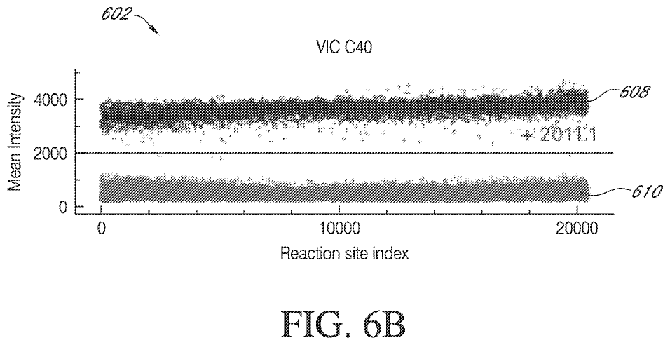

FIGS. 6A and 6B illustrates data visualizations that may be generated and displayed on a user interface to show which reaction sites had fluorescence emissions detected in a 2-plex reaction (using two dyes) according to various embodiments described herein. In data visualization 600 (FIG. 6A), the intensity of FAM dye detection is shown on the y-axis and the reaction site index is shown on the x-axis. With reference to FIG. 6B, in data visualization 602, the intensity of VIC dye detection is shown on the y-axis and the reaction site index is shown on the x-axis. Both data visualizations 600 and 602 clearly show some fluorescent emission detection in a plurality of reaction sites. Indications of intensity values 604 (FIGS. 6A) and 608 (FIG. 6B) show fluorescent emission detection and may be considered positive for fluorescence for their respective dyes. Similarly, indications of intensity values 606 (FIGS. 6A) and 610 (FIG. 6B) show no fluorescent emission detection and may be considered negative for fluorescence for their respective dyes. However, there are outliers from the indications of intensity values 604, 606, 608, and 610 and a threshold at which intensity is considered positive for both FAM and VIC should be determined. Furthermore, as discussed above, spectral interaction between dyes could affect a threshold decision.

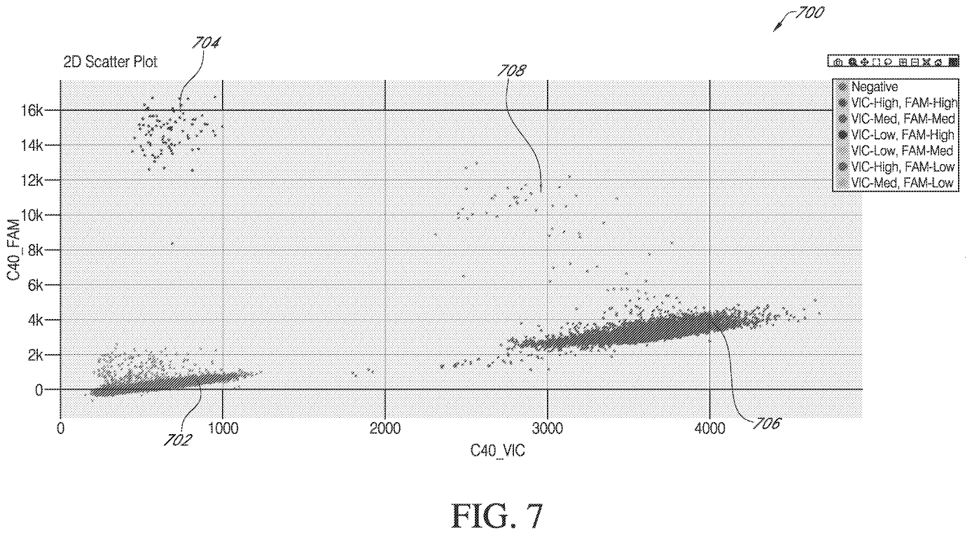

With reference to FIG. 7, a generated 2D scatter plot data visualization showing VIC intensity versus FAM intensity illustrates several clusters of data. Some clusters illustrate negative fluorescence emission, positive FAM fluorescence emission, positive VIC fluorescence emission. Data cluster 702 appears to be negative fluorescence, data cluster 704 appears to be positive FAM, data cluster 706 appears to be positive for VIC, and data cluster 708 may be positive for FAM and VIC. However, there are other sub-clusters and data points that are not clearly positive, which may also be a result of spectral interaction. Further, data clusters appearing to be positive or negative may be affected by spectral crosstalk, which is difficult to determine from a 2D scatter plot data visualization.

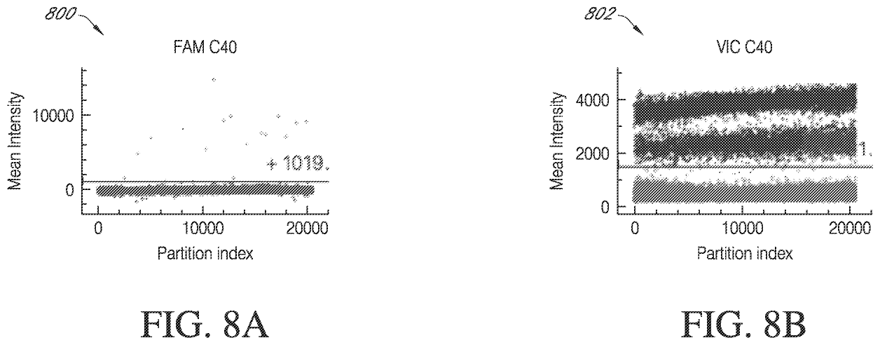

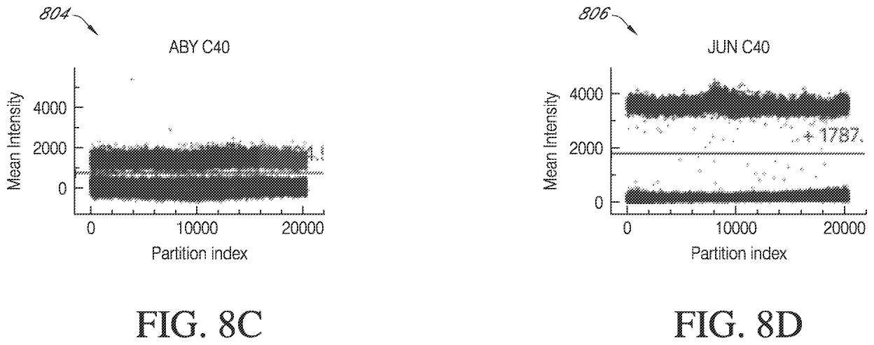

In another example, with reference to FIGS. 8A, 8B, 8C, and 8D, data from a 4-plex reaction (using four dyes) is illustrated. Plots 800 (FIGS. 8A), 802 (FIGS. 8B), 804 (FIG. 8C), and 806 (FIG. 8D) show FAM, VIC, ABY, and JUN dye detection. Similar to FIGS. 6A and 6B, some reaction sites show clearly positive fluorescence, but others are not as certain. Plot 802 even shows two distinct bands of VIC fluorescence detection.

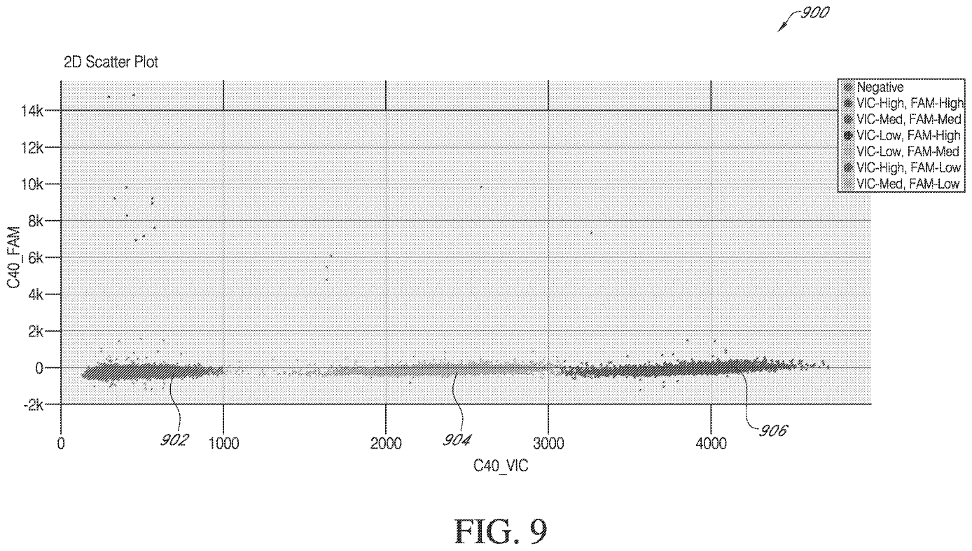

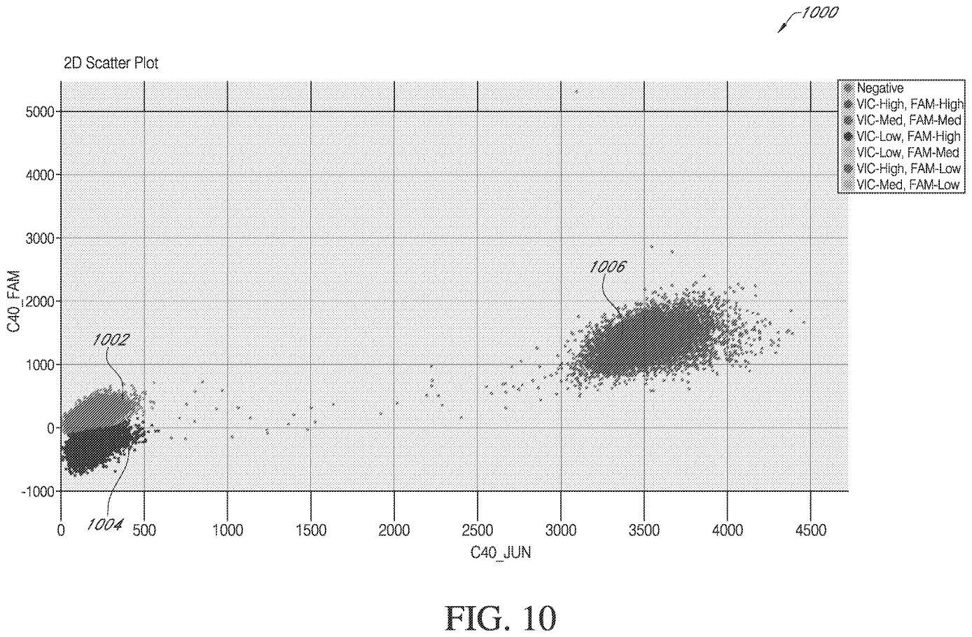

Looking at the same data in 2D scatter plots in FIGS. 9 and 10, comparison between two dyes are visualized. In plot 900, FAM intensity versus VIC intensity is visualized. Data cluster 902 is labeled as negative and data cluster 906 is labeled as positive for VIC. However, it is not clear if data cluster 904 is positive or negative.

As such, according to embodiments described herein, a data visualization that allows flexible cluster calling in multi-dimensional space is needed to give a better ability for a user to investigate spectral and chemical reactions.

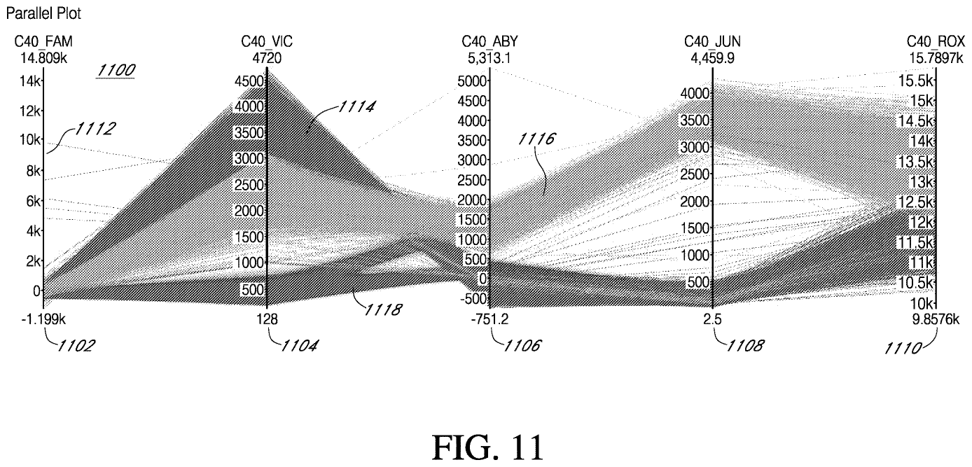

FIG. 11 illustrates a multi-dimensional data visualization 1100 according to various embodiments described herein. Fluorescent emission intensity values of each dye channel are plotted in the multi-dimensional data visualization 1100 so that a comparison can be made, and spectral interference can be more easily determined and accounted for. Fluorescent emission intensity is shown on the y-axis 1112. Data from each optical dye channel is plotted. Axis 1102 shows the fluorescence detection in the FAM channel. Axis 1104 shows the fluorescent emission detection in the VIC channel. Axis 1106 shows the fluorescent emission detection in the ABY channel. Axis 1108 shows the fluorescent emission detection in the JUN channel. Axis 1110 shows the fluorescent emission detection in the ROX channel. The data points in each dye channel are connected by lines to better show the change in intensity values detected in each dye channel. In this way, indications of intensity values detected in each dye channel from the fluorescent emission data including information from all dyes used are visualized.

With reference back to FIG. 9, data cluster 906 corresponds to indications of intensity values 1114. Data cluster 904 corresponds to indications of intensity values 1116. Data cluster 902 corresponds to indications of intensity values 1118.

Axis 1104 showing the indications of intensity values detected in the VIC channel show that some of the data 1116 may have been affected by the true positive JUN detection, as can be seen on the JUN axis 1108. A processor or user may then determine that fluorescent emission data 1116 from a plurality of reaction sites are not positive for VIC because there was spectral interference from the high intensity JUN fluorescence. A user or processor may then select the data that is determined to not be positive of VIC fluorescence based on multi-dimensional data visualization 1100. The labels of corresponding data in 2D scatter plot data visualization 900 is then changed to positive or negative based on the information determined in multi-dimensional data visualization 1100.

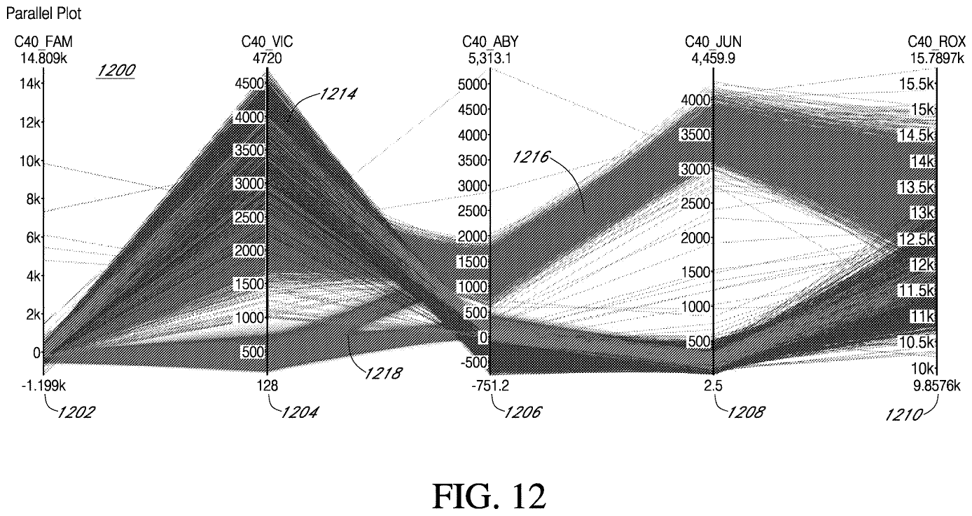

According to various embodiments described herein, FIG. 12 illustrates another exemplary multi-dimensional data visualization 1200. Multi-dimensional data visualization 1200 corresponds to the 2D scatter plot data visualization 1000 with reference back to FIG. 10.

Axis 1202 shows the fluorescence detection in the FAM channel. Axis 1204 shows the fluorescent emission detection in the VIC channel. Axis 1206 shows the fluorescent emission detection in the ABY channel. Axis 1208 shows the fluorescent emission detection in the JUN channel. Axis 1210 shows the fluorescent emission detection in the ROX channel.

Data cluster 1002 corresponds to indications of intensity values 1218. Data cluster 1004 corresponds to indications of intensity values 1214. Data cluster 1006 corresponds to indications of intensity values 1216.

Data cluster 1002 have a label of negative for fluorescence and can be seen as a generally no/low intensity in multi-dimensional data visualization 1200 as indications of intensity values 1218. Indications of intensity values 1218 shows that the negative label appears correct. Data cluster 1004 is labeled as positive for ABY and JUN dyes and is seen as indications of intensity values 1216. Indications of intensity values 1216 appears to show that the positive label is accurate. Data cluster 1004 appears to be negative in 2D scatter plot data visualization 1200, but appears as a separate cluster from data cluster 1002. However, looking at the corresponding indications of intensity values 1214, a user may see that there is spectral crosstalk from VIC in the ABY channel. The high intensity of VIC fluorescence creates crosstalk on the ABY channel. Multi-dimensional data visualization 1200 helps explain why data cluster 1004 is distinct from data cluster 1002.

In various embodiments, the initial labeling of data clusters are user-defined. In other various embodiments, a computing system may provide initial labels based on a predefined threshold. After viewing multi-dimensional data visualization 1200, a user may adjust the label to some data to dynamically change the labeling on 2D scatter plot data visualization 1000 according to various embodiments. Furthermore, the changing of the labels of data will affect the result generated by the computing system according to various embodiments. In an example of a dPCR system, a result generated by the dPCR system, such as concentration of target, is changed based on the labels of the data. In this example, changing a label based on information from a multi-dimensional data visualization also changes the result generated by the dPCR system giving an improved result.

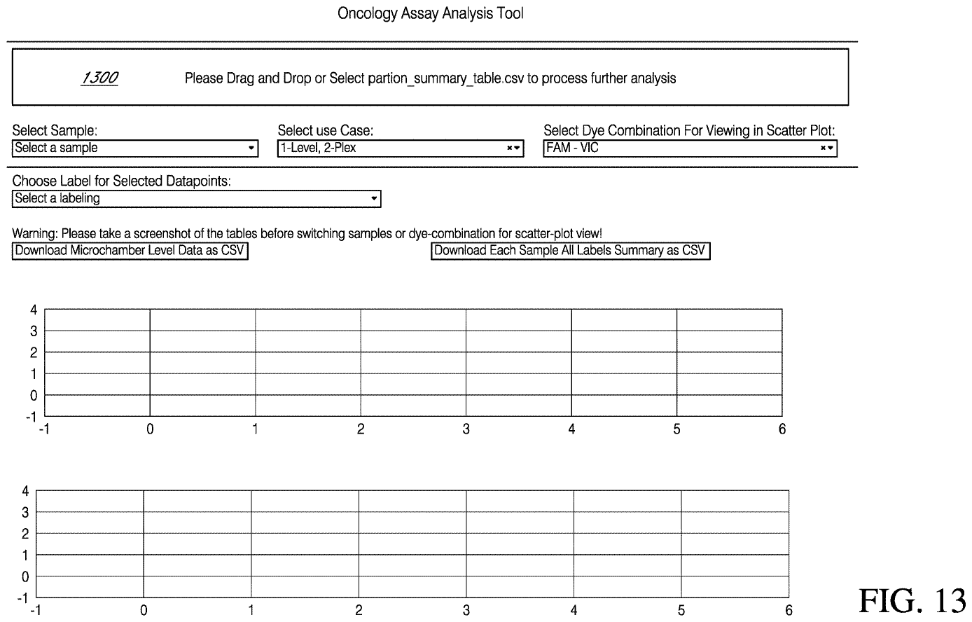

FIG. 13 illustrates a user interface according to various embodiments described herein.

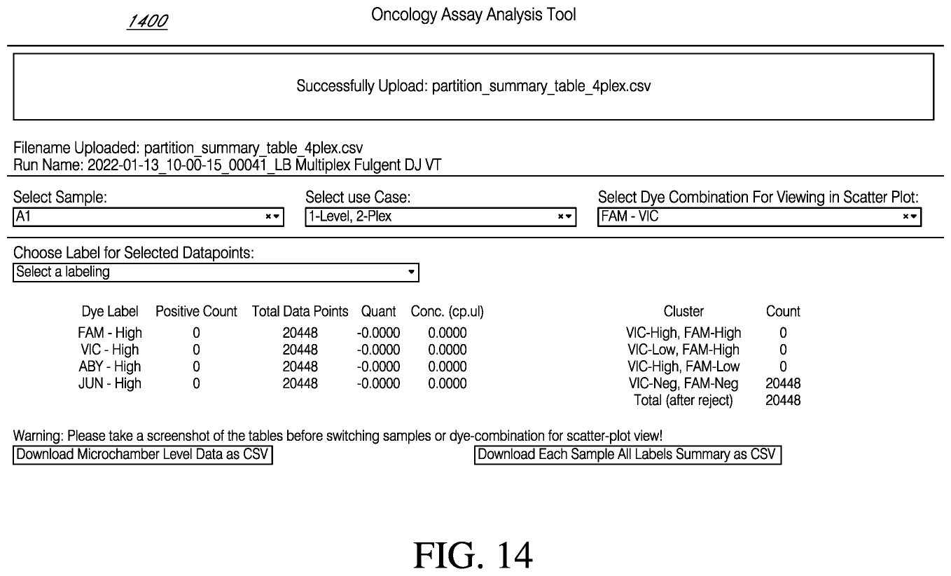

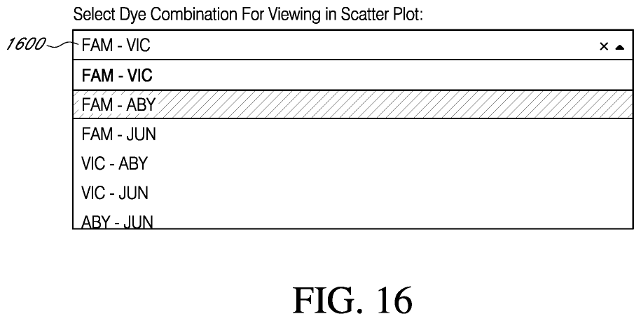

When performing an analysis on a biological sample, a user may access a user interface 1300. In user interface 1300, a user is able to indicate the sample name, for example, the experiment setup of number of dyes used, and which dye combinations the user wants displayed. The user may also have an option to download a file of a summary of all labels for each sample in user interface 1300. For example, a file may be downloaded that includes the intensity values and labels for each reaction site in all samples. Another type of file that may be downloaded is a summary of all labels for each sample. The file may be downloaded as a csv file. An exemplary drop-down menu box 1600 is shown in FIG. 16 to select the dye combination. When the fluorescent emissions are detected, the initial determinations of positive and negative fluorescent detection or displayed as shown in user interface 1400 with reference to FIG. 14. The user may also have an option to download a file of a summary of all labels for each sample in user interface 1400. The file may be downloaded as a csv file.

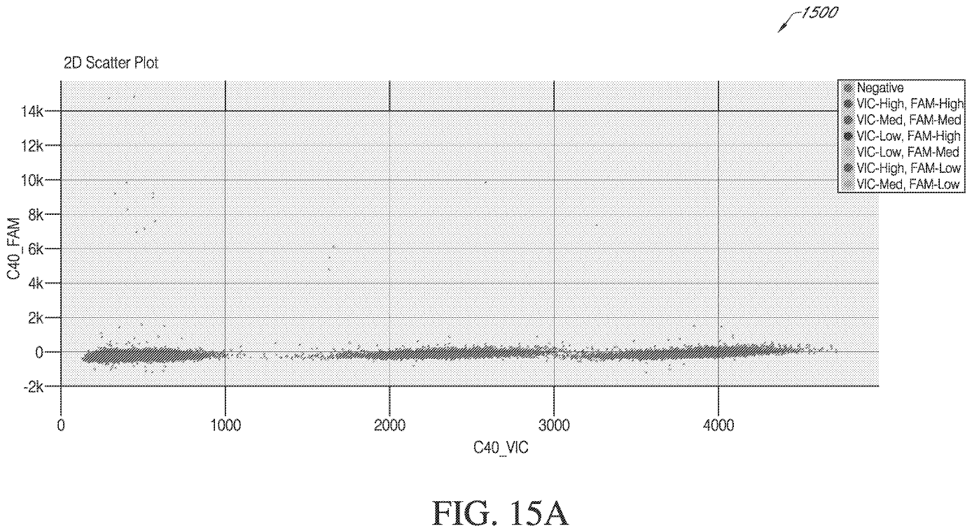

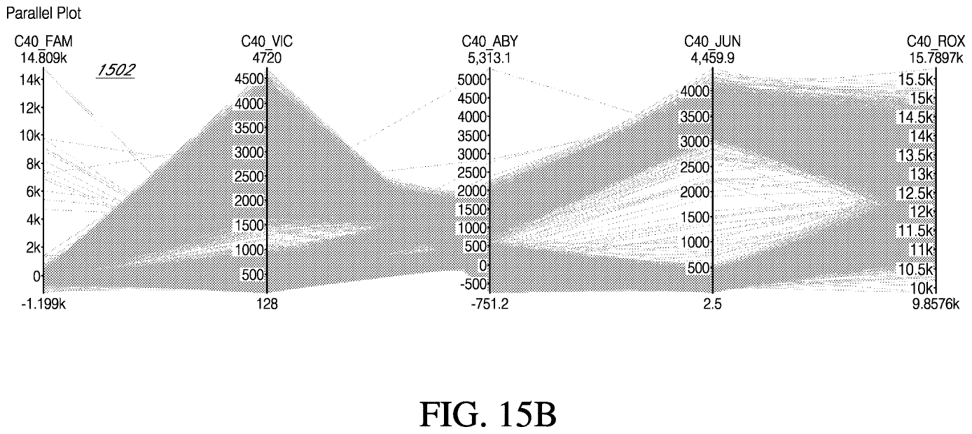

Also displayed on a user interface are a 2D scatter plot data visualization and a multi-dimensional data visualization generated from the fluorescent emission data. Exemplary 2D scatter plot data visualization 1500 and multi-dimensional data visualization 1502 are shown in FIG. 15A and FIG. 15B, respectively.

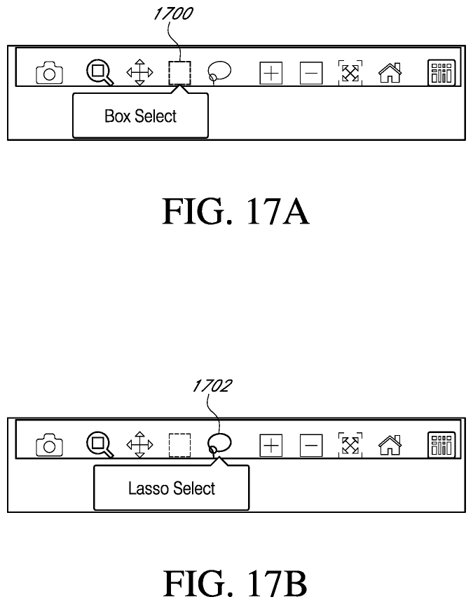

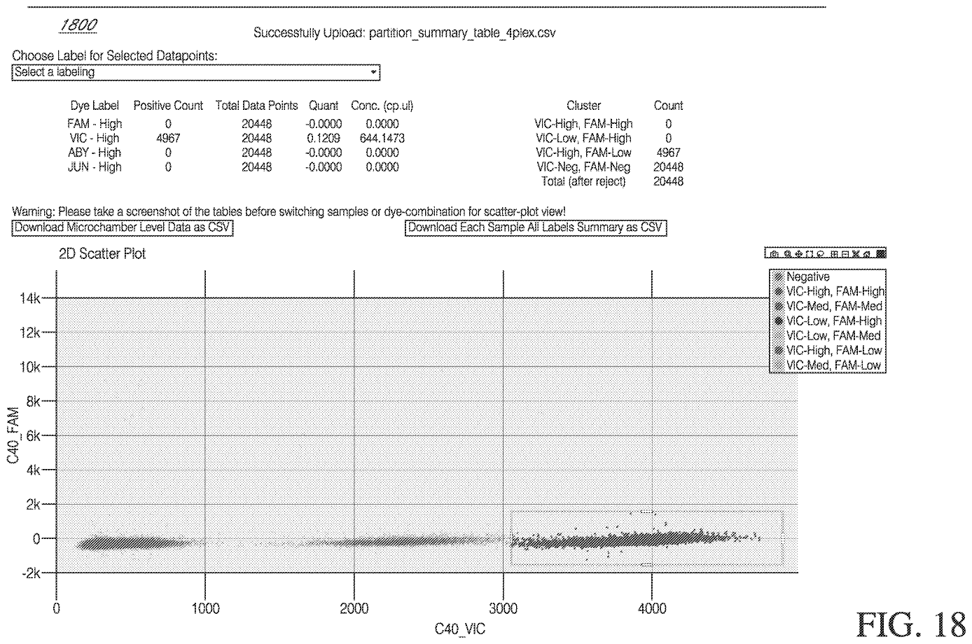

FIGS. 17A and 17B illustrate tools of a user interface according to various embodiments described herein. FIG. 17A illustrates a box selection tool 1700 to quickly select data points. FIG. 17B illustrates a lasso selection tool to draw a shape around data points for selection. The selected data points can change a label such as from positive to negative or negative to positive. The labels can indicate a level of intensity value detected for a dye. For example, labels may include: “VIC High Positive, FAM High Positive”, “VIC Medium Positive, FAM Medium Positive”, “VIC Low, FAM High Positive”, “VIC Low, FAM Medium Positive”, “VIC High Positive, FAM low”, “VIC Medium Positive, FAM Low”, or “VIC Negative, FAM Negative”. However, one skilled in the art would recognize there are other labels that may be used according to various embodiments described herein, such as labels for other dyes or data that is determined to not be included in quantitation result. The selected data points can also be excluded from the result calculation. Data selected by box selection tool 1700 is illustrated in FIG. 18 is an exemplary user interface for a 2D scatter plot data visualization. The user may also have an option to download a file of a summary of all labels for each sample in user interface 1800. The file may be downloaded as a csv file, for example.

A table can also be displayed on the user interface showing the positive counts, total number of data points for each dye, total number of data points for each label, quant values, or concentration values, for example. As mentioned above, multiple dyes may be used.

According to various embodiments, user interface 1200 may also have a tool to download the results in a file, such as a csv file.

Indicators such as colors or labels may also be changed from user interface 1200. The range of intensity displayed can be changed. The display order of the dye channels may also be changed.

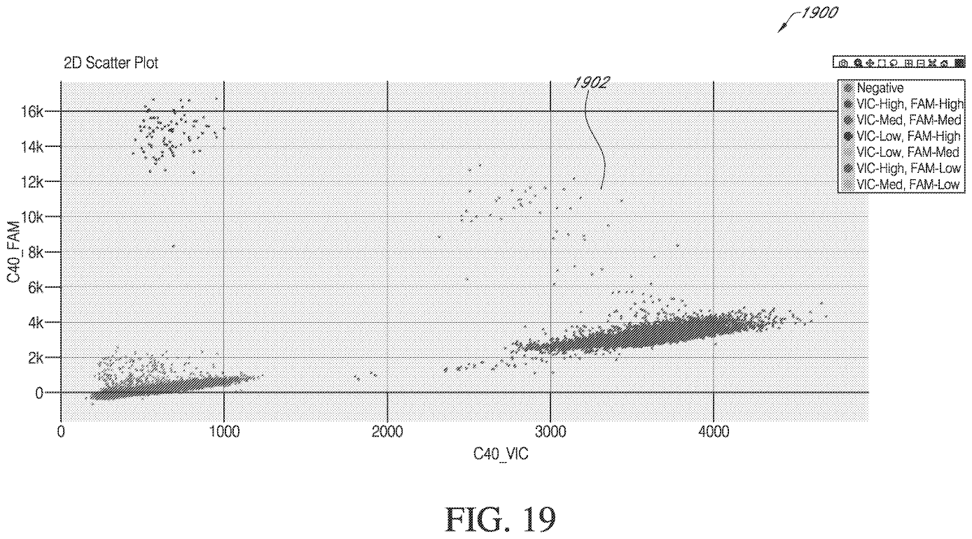

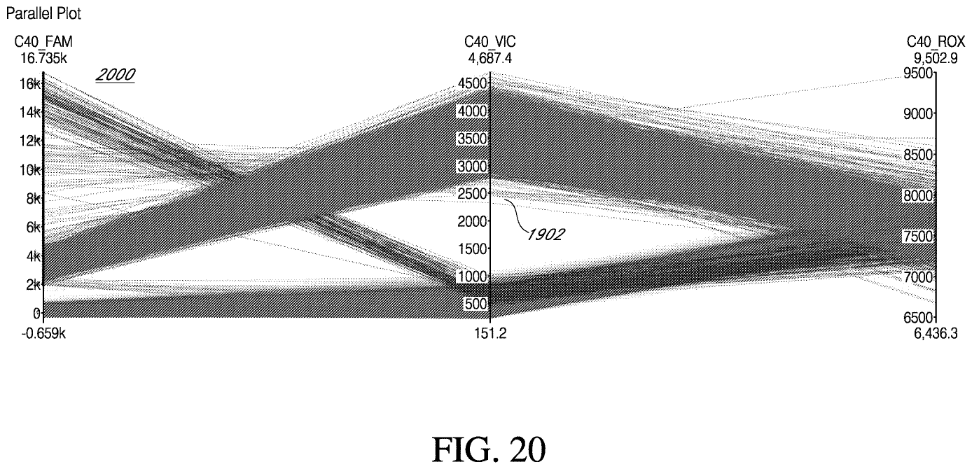

According to various embodiments described herein, FIG. 19 illustrates a 2D scatter plot data visualization. The corresponding multi-dimensional data visualization according to various embodiments described herein is illustrated in FIG. 20. Data subcluster 1902 shows that the data is both VIC and FAM positive. However, from viewing multi-dimensional data visualization 2000, it can be determined that some of the positive VIC and FAM data in 2D scatter plot 1900 may have been affected by fluorescence from other reaction sites.

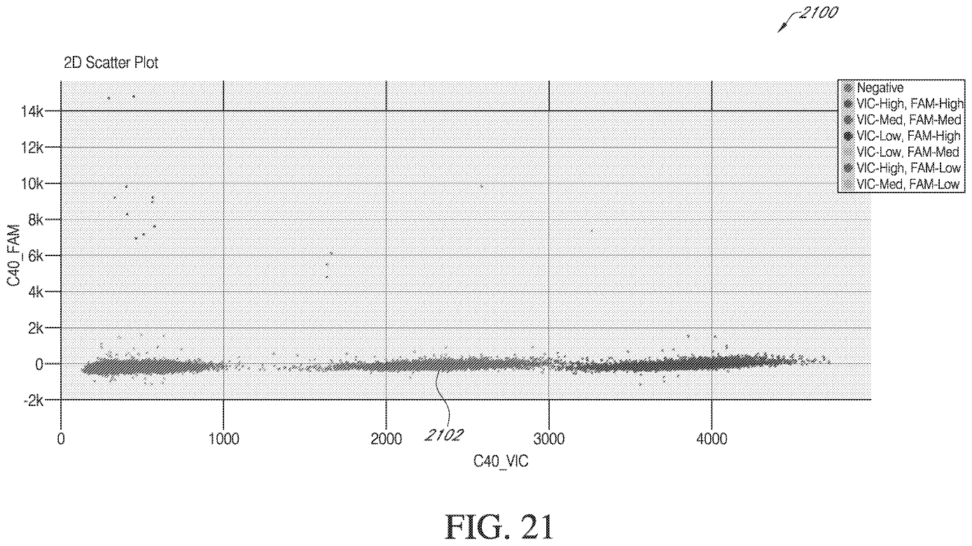

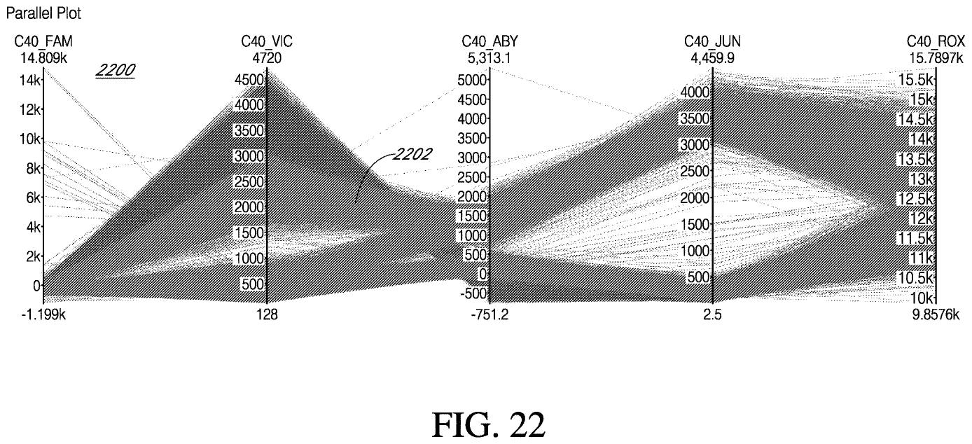

According to various embodiments described herein, FIG. 21 illustrates a 2D scatter plot data visualization 2100 of FAM and VIC detection. The corresponding multi-dimensional data visualization 2200 according to various embodiments described herein is illustrated in FIG. 22. In 2D scatter plot data visualization 2100 shows a label of a data cluster 2102 as negative for VIC even though there seems to be two clusters of data labeled negative for VIC. In reviewing the multi-dimensional data visualization 2200, a user may be able to determine that the second negative data cluster 2102 is probably due to a high intensity JUN detection by looking at the indications of intensity 2202. Thus, the user may be able to confirm the automatic call of the data as negative. In this example, four dyes are used in this multiplex experiment. According to various embodiments, a multidimensional data visualization can use at least two dyes.

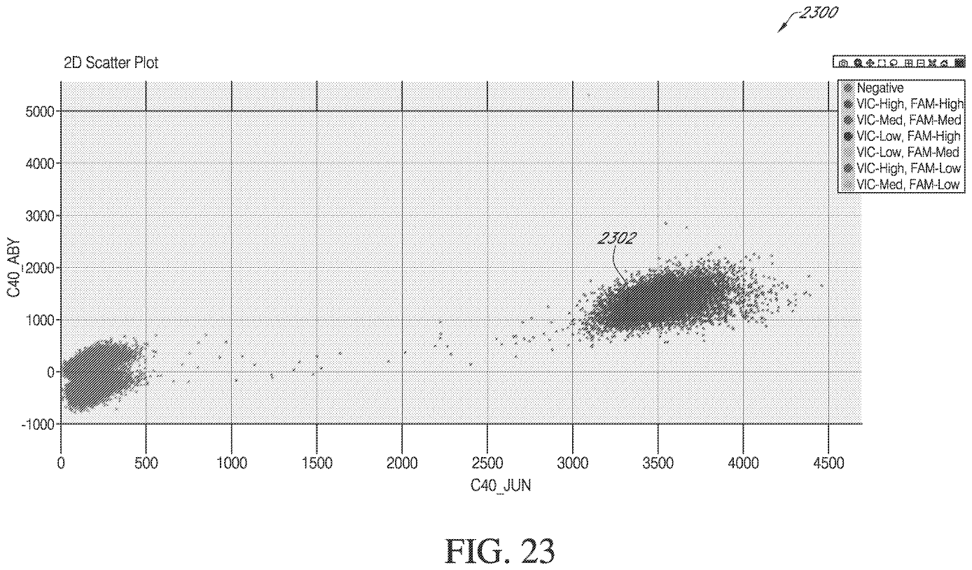

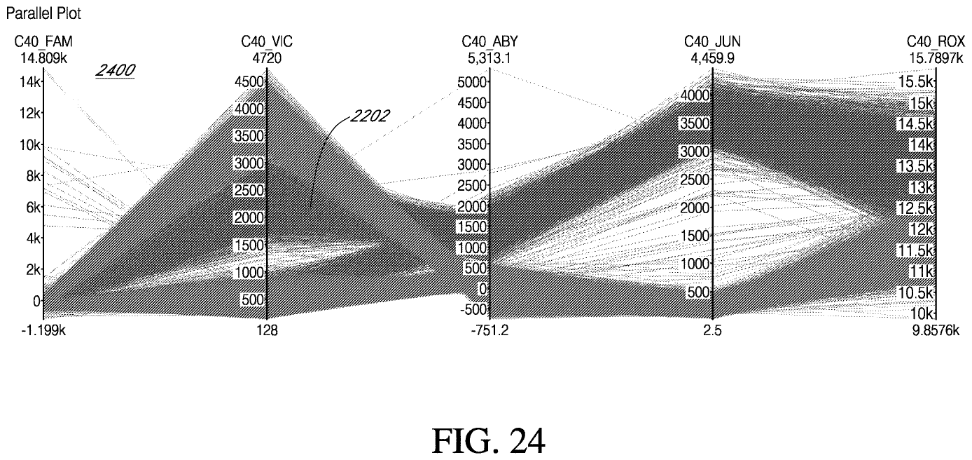

According to various embodiments described herein, FIG. 23 illustrates a 2D scatter plot data visualization 2300. The corresponding multi-dimensional data visualization 2400 according to various embodiments described herein is illustrated in FIG. 24. In the 2D scatter plot data visualization 2300, data cluster 2302 is labeled high for JUN and ABY. According to various embodiments, high fluorescence indicates positive fluorescence detection. For examples, data cluster 2302 may also be labeled positive for JUN and ABY. However, in reviewing the indications of intensity 2402, in the JUN channel for the indications of intensity 2402 are very high. A user is able to see the VIC positive detection may be due to the high intensity of JUN in this data. ABY and JUN positive detection is shown to be true positive detection. In this example, four dyes are used in this multiplex experiment.

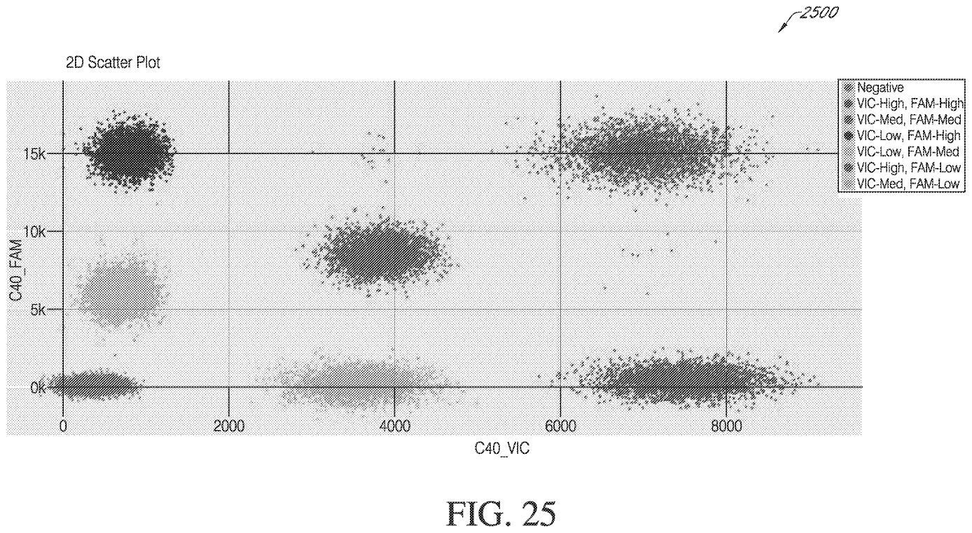

FIG. 25 illustrates a 2D scatter plot data visualization according to various embodiments described herein. In this example, two dyes (FAM and VIC) are used, but two levels of intensity are used. As such, several clusters are generated in 2D scatter plot data visualization 2600. Various combinations of high, medium, and no intensity of the dyes are shown.

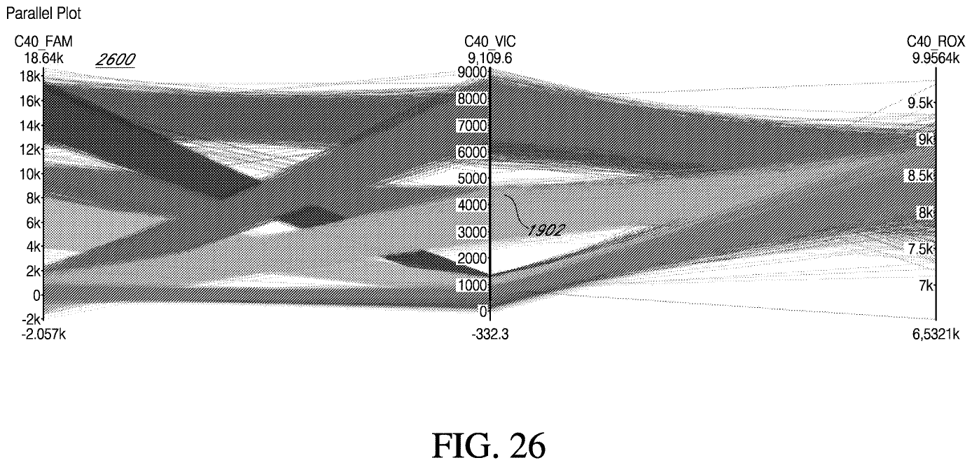

A corresponding multi-dimensional data visualization 2600 is shown in FIG. 26 according to various embodiments discussed herein.

### EXAMPLES

The following numbered examples are embodiments:

1. A computer-implemented method for visualizing dye interaction in a multiplexed biological sample, the method comprising:
receiving fluorescent emission data from each reaction site of a plurality of reaction sites, wherein the plurality of reaction sites include at least a first, second, and third dye;
determining intensity values for at least a first, second, and third dye channel from the fluorescent emission data from each reaction site of the plurality of reaction sites;
displaying, on a user interface, a first set of indications of a plurality of intensity values of the fluorescent emission data for the first, second, and third dye detected in the first dye channel;
displaying, on the user interface, a second set of indications of a plurality of intensity values of the fluorescent emission data for the first, second, and third dye detected in the second dye channel;
displaying, on the user interface, a third set of indications of a plurality of intensity values of the fluorescent emission data for the first, second, and third dye detected in the third dye channel; and adjusting a label of the fluorescent emission data from a reaction site by comparing the first set of indications.

2. The computer-implemented method of example 1, wherein the fluorescent emission data further includes a fourth dye.

3. The computer-implemented method of example 1 or example 2, further comprising:
displaying, on the user interface, a fourth set of indications of a plurality of intensity values of the fluorescent emission data for the first, second, and third dye detected in the fourth dye channel.

4. The computer-implemented method of any one of examples 1 to 3, wherein the label is a positive amplification label.

5. The computer-implemented method of any one of examples 1 to 4, wherein the fluorescent emission data is generated from a polymerase chain reaction (PCR) of a biological sample.

6. The computer-implemented method of any one of examples 1 to 5, wherein the first, second, and third sets of indications are displayed in a plot with a x-axis and a y-axis, wherein the x-axis is dye channels and the y-axis is intensity value.

7. The computer-implemented method of example 6, wherein the plot gives a visualization of the intensity of the first, second, and third dyes in each dye channel.

8. The computer-implemented method of example 6 or example 7, wherein a line is displayed on the user interface connecting the indications of intensities in each dye channel for the fluorescent emission data from the same reaction site.

9. A system for visualizing dye interaction in a multiplexed biological sample, the system comprising:
a plurality of reaction sites, wherein each reaction site includes a biological sample; a detector configured to receive fluorescent emission data from the plurality of reaction sites;
a processor configured to determine intensity values for at least a first, second, and third dye channel from the fluorescent emission data from each reaction site of the plurality of reaction sites; and
a user interface configured to:
   display first set of indications of a plurality of intensity values of the fluorescent emission data for the first, second, and third dye detected in the first dye channel;
   display a second set of indications of a plurality of intensity values of the fluorescent emission data for the first, second, and third dye detected in the second dye channel;
   display a third set of indications of a plurality of intensity values of the fluorescent emission data for the first, second, and third dye detected in the third dye channel.

10. The system of example 1, wherein the fluorescent emission data further includes a fourth dye.

11. The system of example 9 or example 10, wherein the user interface is further configured to:
display a fourth set of indications of a plurality of intensity values of the fluorescent emission data for the first, second, and third dye detected in the fourth dye channel.

12. The system of any one of examples 9 to 11, wherein the label is a positive amplification label.

13. The system of any one of examples 9 to 12, wherein the fluorescent emission data is generated from a polymerase chain reaction (PCR) of a biological sample.

14. The system of any one of examples 9 to 13, wherein the first, second, and third sets of indications are displayed in a plot with a x-axis and a y-axis, wherein the x-axis is dye channels and the y-axis is intensity value.

15. The system of example 14, wherein the plot gives a visualization of the intensity of the first, second, and third dyes in each dye channel.

16. The system of example 14 or example 15, wherein a line is displayed on the user interface connecting the indications of intensities in each dye channel for the fluorescent emission data from the same reaction site.

17. A computer-readable medium encoded with computer-readable instructions, which when executed by a processor of a computer, causes the computer to carry out the method of any one of the examples 1 to 8.

18. A system comprising a processor, and a storage medium storing instruction, which when executed by a processor, causes the system to carry out the method of any one of examples 1 to 8.

Although the present invention has been described with respect to certain exemplary embodiments, examples, and applications, it will be apparent to those skilled in the art that various modifications and changes may be made without departing from the invention.

What is claimed is:

1. A computer-implemented method for visualizing dye interaction in a multiplexed biological sample, the method comprising:
receiving fluorescent emission data from each reaction site of a plurality of reaction sites, wherein the plurality of reaction sites include at least a first, second, and third dye;
determining intensity values for at least a first, second, and third dye channel from the fluorescent emission data from each reaction site of the plurality of reaction sites;
displaying, on a user interface, a first set of indications of a plurality of intensity values of the fluorescent emission data for the first, second, and third dye detected in the first dye channel;
displaying, on the user interface, a second set of indications of a plurality of intensity values of the fluorescent emission data for the first, second, and third dye detected in the second dye channel;
displaying, on the user interface, a third set of indications of a plurality of intensity values of the fluorescent emission data for the first, second, and third dye detected in the third dye channel; and
adjusting a label of the fluorescent emission data from a reaction site by comparing the first set of indications.

2. The computer-implemented method of claim 1, wherein the fluorescent emission data further includes a fourth dye.

3. The computer-implemented method of claim 2, further comprising:
displaying, on the user interface, a fourth set of indications of a plurality of intensity values of the fluorescent emission data for the first, second, and third dye detected in the fourth dye channel.

4. The computer-implemented method of claim 1, wherein the label is a positive amplification label.

5. The computer-implemented method of claim 1, wherein the fluorescent emission data is generated from a polymerase chain reaction (PCR) of a biological sample.

6. The computer-implemented method of claim 1, wherein the first, second, and third sets of indications are displayed in a plot with a x-axis and a y-axis, wherein the x-axis is dye channels and the y-axis is intensity value.

7. The computer-implemented method of claim 6, wherein the plot gives a visualization of the intensity of the first, second, and third dyes in each dye channel.

8. The computer-implemented method of claim 7, wherein a line is displayed on the user interface connecting the indications of intensities in each dye channel for the fluorescent emission data from the same reaction site.

9. A system for visualizing dye interaction in a multiplexed biological sample, the system comprising:
a plurality of reaction sites, wherein each reaction site includes a biological sample;
a detector configured to receive fluorescent emission data from the plurality of reaction sites;
a processor configured to determine intensity values for at least a first, second, and third dye channel from the fluorescent emission data from each reaction site of the plurality of reaction sites; and
a user interface configured to:
display first set of indications of a plurality of intensity values of the fluorescent emission data for the first, second, and third dye detected in the first dye channel;
display a second set of indications of a plurality of intensity values of the fluorescent emission data for the first, second, and third dye detected in the second dye channel;
display a third set of indications of a plurality of intensity values of the fluorescent emission data for the first, second, and third dye detected in the third dye channel.

10. The system of claim 9, wherein the fluorescent emission data further includes a fourth dye.

11. The system of claim 10, wherein the user interface is further configured to:
display a fourth set of indications of a plurality of intensity values of the fluorescent emission data for the first, second, and third dye detected in the fourth dye channel.

12. The system of claim 9, wherein the label is a positive amplification label.

13. The system of claim 9, wherein the fluorescent emission data is generated from a polymerase chain reaction (PCR) of a biological sample.

14. The system of claim 9, wherein the first, second, and third sets of indications are displayed in a plot with a x-axis and a y-axis, wherein the x-axis is dye channels and the y-axis is intensity value.

15. The system of claim 14, wherein the plot gives a visualization of the intensity of the first, second, and third dyes in each dye channel.

16. The system of claim 14, wherein a line is displayed on the user interface connecting the indications of intensities in each dye channel for the fluorescent emission data from the same reaction site.

17. A computer-readable medium encoded with computer-readable instructions, which when executed by a processor of a computer, causes the computer to carry out the method of claim 1.

18. A system comprising a processor, and a storage medium storing instruction, which when executed by a processor, causes the system to carry out the method of claim 1.
